(19) United States
(12) Patent Application Publication
(10) Pub. No.: US 2021/0403642 A1
(43) Pub. Date: Dec. 30, 2021

(54) AZLACTONE FUNCTIONALIZED SUBSTRATES FOR CONJUGATION OF BIOMOLECULES

(71) Applicant: Robert Bosch GmbH, Stuttgart (DE)

(72) Inventors: Nadezda Fomina, Redwood City, CA (US); Autumn Maruniak, Fremont, CA (US); Christopher Johnson, San Carlos, CA (US); Habib Ahmad, Sunnyvale, CA (US); Sam Kavusi, Menlo Park, CA (US)

(21) Appl. No.: 17/342,146

(22) Filed: Jun. 8, 2021

Related U.S. Application Data
(62) Division of application No. 15/169,008, filed on May 31, 2016, now abandoned.

(51) Int. Cl. C08G 65/333; C08L 33/12; C08L 23/00; C08H 1/00; C08B 37/00; C08G 73/02; C08L 71/02; C08L 5/00; C09D 171/02; C08L 33/26; C09D 171/00

(52) U.S. Cl. CPC C08G 65/33396; C08L 33/12; C08L 23/00; C08H 1/00; C08B 37/00; C08G 73/0233; C08L 71/02; C08L 5/00; C09D 171/02; C08L 33/26; C09D 171/00; C08G 73/024

(57) ABSTRACT

A bifunctional polymer is functionalized at one end with an azlactone end group to conjugate biomolecules of interest, and is functionalized at another end with an azide anchor group to attach the polymer to a substrate. Methods of making the bifunctional polymer are also provided. A coated substrate includes the bifunctionalized polymers on the surface of a substrate. Methods of making the coated substrate are also provided. A microarray includes a plurality of discrete regions, each region including the coated substrate.

### CROSS-REFERENCE TO RELATED APPLICATIONS

The present application is a divisional of and claims priority to U.S. patent application Ser. No. 15/169,008, filed on May 31, 2016.

### FIELD OF THE INVENTION

Aspects of the present invention relate to a bifunctional polymer and methods of making the bifunctional polymer, where the bifunctional polymer is functionalized at one end with an azlactone group to conjugate biomolecules of interest, and functionalized at another end with an azide anchor group to attach the polymer to a substrate, such that azlactone functionality remains intact under conditions where the azide group is reactive. Aspects of the present invention also relate to a coated substrate that includes such bifunctionalized polymers on the surface of the substrate, and methods of making the coated substrate. Aspects of the present invention also relate to a microarray that includes a plurality of discrete spots, each spot including the coated substrate on the surface of the microarray.

### BACKGROUND

In measurement assays or platforms, such as protein and antibody microarrays, achieving high fidelity (e.g., sensitivity and specificity) of the target molecule is of great importance. The samples in assays can be biomolecules such as nucleic acids, proteins, biological cells, and small molecules, along with other human bodily fluids such as blood, serum, saliva, and urine, and also consumables such as milk, baby food, or water. Regardless of the sample, efficient conjugation of the target molecule to the substrate of the assay can help achieve higher fidelity in these assays.

Activated esters (N-hydroxysuccinimide (NHS), maleimide, fluorophenyl), carbamates, carbonates, epoxides, and aldehydes are functional groups that are commonly used in assays for conjugation of biomolecules. However, these functional groups suffer from low hydrolytic stability. Low hydrolytic stability can lead to suboptimal efficiency of conjugation of the biomolecules due to the competing hydrolysis reaction, which results in a lower amount of conjugated biomolecules and non-uniformity of conjugated biomolecules.

Azlactone groups are known to react with strong nucleophiles, such as primary amines, alcohols, and thiols, via a ring-opening addition reaction, while showing good resistance to water hydrolysis at a neutral pH (see Carter, H. E., Chapter 5: “Azlactones,” Organic Reactions, John Wiley & Sons, 3:198-239 (1946)). In addition to higher stability, azlactone-functionalized surfaces can be stored longer under ambient conditions, while surfaces containing activated esters, epoxides, and aldehydes have to be stored in moisture-free conditions (vacuum-sealed packaging, freezer) and utilized immediately upon exposure to ambient conditions.

“Azlactone” can be represented by a 6-membered ring:

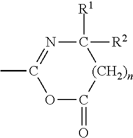

where R₁ and R₂ independently can be hydrogen, an alkyl group having 1 to 14 carbon atoms, a cycloalkyl group having 3 to 14 carbon atoms, an aryl group having 5 to 12 ring atoms, an arenyl group having 6 to 26 carbon atoms and 0 to 3 sulfur, nitrogen, and nonperoxidic oxygen heteroatoms, or R₁ and R₂ taken together with the carbon to which they are joined can form a carbo-cyclic ring containing 4 to 12 ring atoms; and n is an integer 0 or 1.

If n is 0, then azlactone can be represented by a 5-membered ring:

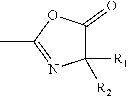

where R₁ and R₂ are defined as above.

Due to their advantageous and unique properties, methods of preparing azlactone functionalized polymers for use in protein conjugation are known. U.S. Pat. No. 5,321,095 discloses a method of producing azlactone-activated polyalkylene oxides, including polyethylene glycol (PEG), for the purpose of preparing protein-PEG conjugates. U.S. Pat. Nos. 4,485,236 and 5,013,795 also disclose methods of preparing azlactone-containing polymers via radical processes and their use for protein conjugation.

Azlactone groups have been utilized in chromatography. Rasmussen et al., “Crosslinked, hydrophilic, azlactone-functional polymeric beads: A two-step approach,” Reactive Polymers, 16(2):199-212 (1991), discloses the application of azlactone-functionalized polymer beads for chromatography, where acrylamide-based polymer beads are produced via radical polymerization, and azlactone groups are introduced via cyclodehydration of pendant acylamino acid groups using acetic anhydride. Coleman et al., “Immobilization of Protein A at High Density on Azlactone-functional Polymeric Beads and Their Use in Affinity Chromatography,” Journal of Chromatography, 512:345-63 (1990), discloses highly cross-linked copolymer beads with protein immobilized on their surfaces, prepared by an inverse-phase polymerization process from methylene-bis-acrylamide and vinyldimethyl azlactone, for use in affinity chromatography.

Azlactone groups have also been utilized in cell and enzyme immobilization substrates. Buck et al., “Chemical Modification of Reactive Multilayered Films Fabricated from Poly(2-alkenyl azlactone)s: Design of Surfaces That Prevent or Promote Mammalian Cell Adhesion and Bacterial Biofilm Growth,” Biomacromolecules, 10(6):1564-74 (2009), discloses reactive polymer films that can be functionalized to either prevent or promote an attachment and growth of cells through layer-by-layer assembly of polyamine and poly(2-vinyl-4,4′-dimethylazlactone) (PVDMA). The reaction between amino groups of polyamine with azlactone functionality of PVDMA results in covalent cross-linking of films. The unreacted azlactone groups of PVDMA are further utilized to attach hydrophilic or hydrophobic moieties, thus promoting either cell adhesion or cell repulsion from the surface. Cullen et al., “Surface-Anchored Poly(2-vinyl-4,4-dimethyl azlactone) Brushes as Templates for Enzyme Immobilization,” Langmuir, 24(23):13701-09 (2008), discloses growing PVDMA brushes from initiators anchored to the surface of glass via atom transfer radical polymerization.

Azlactone groups have also been utilized in medical implants. U.S. Pat. No. 5,292,514 discloses preparation of polymeric and oligomeric vinyldimethyl azlactones via radical polymerization and their use as coatings for mammalian body implants.

U.S. Pat. No. 4,981,933 discloses preparation of azlactone copolymers from unsaturated polymerizable azlactone polymer (such as vinyl azlactone) and vinylbenzylhalide, thus having the respective reaction functionalities of these two moieties.

U.S. Pat. No. 5,344,701 discloses several methods of introducing azlactone functionality onto existing supports for coupling of bioreagents. Those methods include high-energy radiation to generate free radicals that subsequently react with vinyl-azlactone, chemical crosslinking of azlactone monomers to form a film on the surface, and dispersion polymerization to produce functional particles within pores of the support.

### SUMMARY

A summary of certain example embodiments of the present invention is set forth below. It should be understood that these aspects are presented merely to provide the reader with a brief summary of these certain embodiments and that these aspects are not intended to limit the scope of the present invention. Indeed, this invention can encompass a variety of aspects that may not be set forth below.

Azlactone functionalized surfaces in measurement platforms or assays can potentially produce spots with superior brightness and uniformity. Because of higher stability of azlactone-functionalized surfaces to water hydrolysis, reproducibility of spotting can potentially be improved as well. But to utilize azlactone groups in measurement platforms or assays, the azlactone functionalized polymers should be immobilized to a surface or substrate without disrupting the functionality of the azlactone groups. In other words, a second functional group must attach the azlactone functionalized polymers to the surface or substrate, but should not interfere with the ability of the azlactone groups to conjugate biomolecules of interest.

Thus, example embodiments of the present invention provide a polymer functionalized with both azlactone groups to conjugate biomolecules and also attachment groups to immobilize the azlactone functionalized polymer onto a substrate to create a biocompatible surface, where azlactone functionality remains intact under the conditions that render the attachment group reactive. According to example embodiments, PEG is functionalized with azlactone groups for conjugation of biomolecules and with azide groups for attachment to a substrate. Azide and azlactone groups are chemically orthogonal, such that azlactone functionality will remain intact under the conditions that render the azide groups reactive. Example embodiments of the present invention also provide methods of making such biocompatible surfaces and incorporating such biocompatible surfaces as substrates in measurement platforms, such as protein and antibody microarrays.

Example embodiments of the present invention provide azlactone functionalized polymers that can be anchored to a surface or substrate, where the resulting azlactone functionalized surface or substrate can be used in measurement assays, such as protein and antibody microarrays, for more efficient conjugation of biomolecules. Such functionalized surfaces can be utilized in biosensor systems as described in U.S. patent application Ser. Nos. 14/792,553, 14/792,576, 14/792,541, 14/792,569, and 14/792,530, which are hereby incorporated herein by reference in their entireties.

According to example embodiments, a bifunctional polymer includes: (a) an anchor group selected from a group consisting of azide, carboxylic acid, thiol, amine, hydroxyl, hydrazine, silyl, phosphonate, alkyne, catechol, and lysine; (b) a polymer block that includes one or more first polymers, the one or more first polymers including PEG or polysaccharide; (c) a linker group selected from a group consisting of phenyl, vinyl, benzyl, and alkyl; and (d) an azlactone end group containing R₁ and R₂, where R₁ and R₂ are each independently selected from a group consisting of hydrogen, alkyl, and aryl.

In some example embodiments, the one or more first polymers are copolymers with a second polymer, where the second polymer is selected from a group consisting of polylysine, polyoxazoline, polymethylmethacrylate, poly-N-isopropylacrylamide, polydopamine, polyalkane, and N-substituted glycine polymer.

According to example embodiments, a coated substrate includes: (a) a substrate; and (b) a polymer layer attached to the substrate, where the polymer layer includes one or more first polymers, one or more azlactone functional groups attached to a first end of each of the one or more first polymers, and one or more azide groups attached to a second end of each of the one or more first polymers, the one or more azide groups attaching the polymer layer to the substrate.

In some example embodiments, the substrate is selected from a group consisting of glass, silica, plastic, carbon, metal, and metal oxide.

In some example embodiments, the polymer layer is linear polymer, multiarm polymer, brush polymer, or nanoparticles.

In some example embodiments, the one or more first polymers are PEG or polysaccharide.

In some example embodiments, the one or more first polymers are copolymers with a second polymer, where the second polymer is selected from a group consisting of polylysine, polyoxazoline, polymethylmethacrylate, poly-N-isopropylacrylamide, polydopamine, polyalkane, and N-substituted glycine polymer.

According to example embodiments, a microarray includes a plurality of discrete regions, where each discrete region includes a coated substrate, the coated substrate including: (a) a substrate; and (b) a polymer layer that is attached to the substrate and that includes one or more polymers, one or more azlactone functional groups attached to a first end of each of the one or more polymers and one or more azide groups attached to a second end of each of the one or more polymers, where the one or more azide groups attach the polymer layer to the substrate.

According to example embodiments, a method of preparing a bifunctional polymer includes: (a) providing a polymer with one or more carboxylic acid groups or activated ester groups attached to a first end of the polymer and one or more azide groups attached to a second end of the polymer; (b) converting at least one carboxylic acid group or activated ester group into an aryl or vinyl halide group; and (c) attaching vinyldialkyl azlactone to the aryl or vinyl halide group.

In some example embodiments, the polymer is PEG.

In some example embodiments, the vinyldialkyl azlactone is attached through a coupling reaction using a palladium catalyst, one or more solvents, and a base.

In some example embodiments, the palladium catalyst is selected from a group consisting of palladium (II) quinoline-8-carboxylate, palladium (II) chloride, palladium (II) bromide, palladium (II) acetate, palladium (II) acetoacetate, bis(dibenzylideneacetone)palladium(0), tris(dibenzylideneacetone)dipalladium(0), palladium (II) trifluoroacetate, allylpalladium (II) chloride dimer, bis(triphenylphosphine)palladium(II) dichloride, dichlorobis(tricyclohexylphosphine)palladium(II), and [1,1′-bis(di-tert-butylphosphino)ferrocene]dichloropalladium(II).

In some example embodiments, the one or more solvents are selected from a group consisting of dimethylformamide, dimethylsulfoxide, toluene, tetrahydrofuran, dioxane, dichloromethane, acetonitrile, and alcohol.

In some example embodiments, the base is selected from a group consisting of potassium carbonate, sodium carbonate, triethylamine, N,N-diisopropylethylamine, 4-(dimethylamino)pyridine, potassium tert-butoxide, and sodium tert-butoxide.

According to example embodiments, there is provided a method of attaching to a substrate a bifunctional polymer, the bifunctional polymer including a polymer, one or more azlactone functional groups attached to a first end of the polymer, and one or more azide groups attached to a second end of the polymer, the method including: (a) providing a substrate containing alkyne functional groups; (b) providing a mixture including the bifunctional polymer, one or more solvents, a catalyst, a base, and a reducing agent; and (c) contacting the substrate with the mixture.

In some example embodiments, the polymer is PEG.

In some example embodiments, the one or more solvents are selected from a group consisting of dimethylformamide, dimethylsulfoxide, toluene, tetrahydrofuran, dioxane, acetonitrile, and water.

In some example embodiments, the catalyst is a copper (II) salt or copper (I) salt.

In some example embodiments, the catalyst is selected from a group consisting of copper sulfate, copper bromide, and copper iodide.

In some example embodiments, the catalyst is a ruthenium catalyst.

In some example embodiments, the catalyst is selected from a group consisting of pentamethylcyclopentadienylbis(triphenylphosphine)ruthenium(II) chloride, pentamethylcyclopentadienyl(cyclooctadienyl)ruthenium(II) chloride, and pentamethylcyclopentadienyl(norbornadiene)ruthenium(II) chloride.

In some example embodiments, the base is selected from a group consisting of triethylamine, N,N-diisopropylethylamine, 4-(dimethylamino)pyridine, pyridine, quinolone, phenanthroline, and imidazole.

In some example embodiments, the reducing agent is selected from a group consisting of sodium ascorbate, tris(triazole)amine, and hydroquinones.

According to example embodiments, a method of isolating biomolecules of interest includes: (a) providing a functionalized substrate, where the functionalized substrate includes a substrate, one or more polymers, one or more azlactone groups attached to a first end of each of the one or more polymers, and one or more azide groups attached to a second end of each of the one or more polymers, the one or more azide groups thereby attaching each of the one or more polymers to the substrate; (b) providing an aqueous solution, where the aqueous solution contains the biomolecules of interest; and (c) contacting the functionalized substrate with the aqueous solution for a period of time, where during the period of time, the biomolecules of interest attach to the azlactone groups of the substrate.

In some example embodiments, the aqueous solution includes an additive, where the additive is selected from a group consisting of glycerol, oligoethylene glycol, polyethylene glycol, surfactants, polyvinylalcohol, sugars, organic solvents, and inorganic salts.

In some example embodiments, a pH level of the aqueous solution is in a range of from about 2 to about 10.

In some example embodiments, contacting the substrate with the aqueous solution is achieved by jet printing, pin printing, quill printing, biological laser printing, capillary-based fluidics, or immersion.

These and other features, aspects, and advantages of the present invention will become better understood when the following detailed description of certain exemplary embodiments is read with reference to the accompanying drawings in which like characters represent like parts throughout the drawings.

### BRIEF DESCRIPTION OF THE DRAWINGS

FIG. 1 illustrates a bifunctional polymer according to an example embodiment of the present invention.

FIG. 2 illustrates a coated substrate according to an example embodiment of the present invention.

FIG. 3 illustrates a microarray according to an example embodiment of the present invention.

FIG. 4 compares the fluorescence intensity of Green Fluorescent Protein (GFP) spots on NETS- and Azlactone-functionalized substrates (average of 10 spots).

FIG. 5A illustrates representative examples of GFP spot morphology obtained on NHS-functionalized substrates. FIG. 5B illustrates representative examples of GFP spot morphology obtained on azlactone-functionalized substrates.

FIG. 6 compares the fluorescence intensity of Immunoglobulin G (IgG) conjugated onto the surface of PEG-coated slides functionalized with azlactone groups and NHS groups.

FIG. 7 illustrates conversion of NHS-functionalized surfaces into azlactone-functionalized surfaces.

FIG. 8 illustrates conversion of aldehyde-functionalized surfaces into azlactone-functionalized surfaces.

### DETAILED DESCRIPTION

Example embodiments of the present invention provide a bifunctional polymer, such as PEG, functionalized at one end with an azlactone end group to conjugate biomolecules of interest, and functionalized at another end with an azide anchor group to attach the polymer to a substrate. Functionalizing a polymer with two groups that are chemically orthogonal, like azlactone and azide, allows azlactone functionality for conjugation of biomolecules to remain intact under conditions where the azide group is reactive for attaching the polymer to the substrate. Example embodiments of the present invention provide a coated substrate that incorporates such bifunctionalized polymers on the surface of a substrate, where the azide group attaches the polymer to the substrate, leaving the azlactone group free and active to conjugate biomolecules. Example embodiments of the present invention provide a microarray that incorporates such a coated substrate on the surface of the microarray, where the microarray contains a plurality of discrete spots, each spot containing the bifunctionalized polymers.

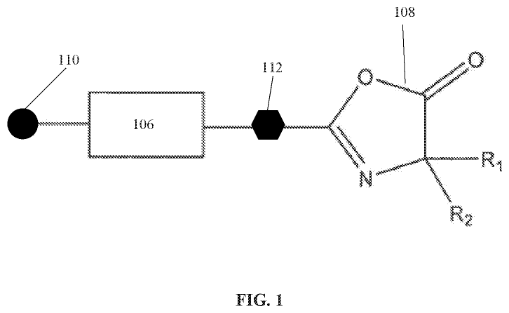

FIG. 1 shows a bifunctional polymer according to an example embodiment that includes an anchor group 110 selected from a group consisting of azide, carboxylic acid, thiol, amine, hydroxyl, hydrazine, silyl, phosphonate, alkyne, catechol, and lysine; a polymer block 106 that includes one or more first polymers, which can be PEG or polysaccharide; a linker group 112 selected from a group consisting of phenyl, vinyl, benzyl, and alkyl; and an azlactone end group 108 that includes R₁ and R₂, where R₁ and R₂ are each independently selected from a group consisting of hydrogen, alkyl, and aryl. In another example embodiment, the one or more first polymers can be copolymers with a second polymer, where the second polymer is selected from a group consisting of polylysine, polyoxazoline, polymethylmethacrylate, poly-N-isopropylacrylamide, polydopamine, polyalkane, and N-substituted glycine polymer.

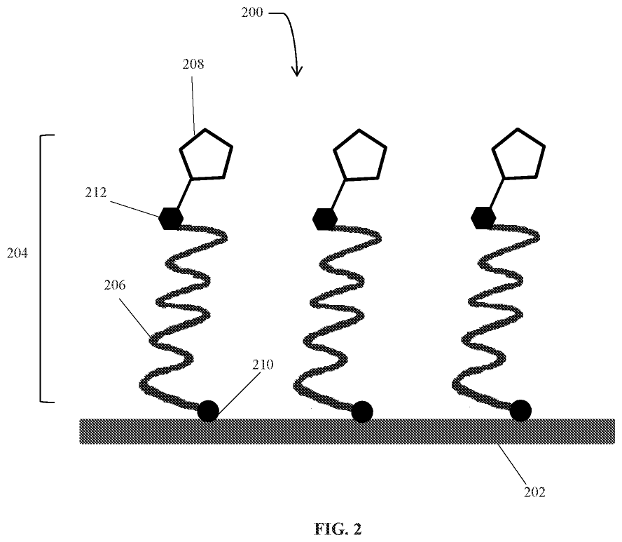

FIG. 2 shows a coated substrate 200 according to an example embodiment, the coated substrate 200 including substrate 202 and polymer layer 204 attached to substrate 202, where polymer layer 204 includes one or more first polymers 206, one or more azlactone functional groups 208 attached to a first end of each of the one or more first polymers 206 through a linker group 212, and one or more azide groups 210 attached to a second end of each of the one or more first polymers 206, where the one or more azide groups 210 attach polymer layer 204 to substrate 202.

In example embodiments, substrate 202 can be glass, silica, plastic, carbon, metal, or metal oxide. As shown in FIG. 2, polymer layer 204 can be made of linear polymers, but in other example embodiments, polymer layer 204 can also be made of multiarm polymers, brush polymers, or nanoparticles. In some example embodiments, the one or more first polymers 206 can be PEG or polysaccharide, and in other example embodiments, the one or more first polymers 206 can be copolymers with a second polymer, where the second polymer can be polylysine, polyoxazoline, polymethylmethacrylate, poly-N-isopropylacrylamide, polydopamine, polyalkane, or N-substituted glycine polymer.

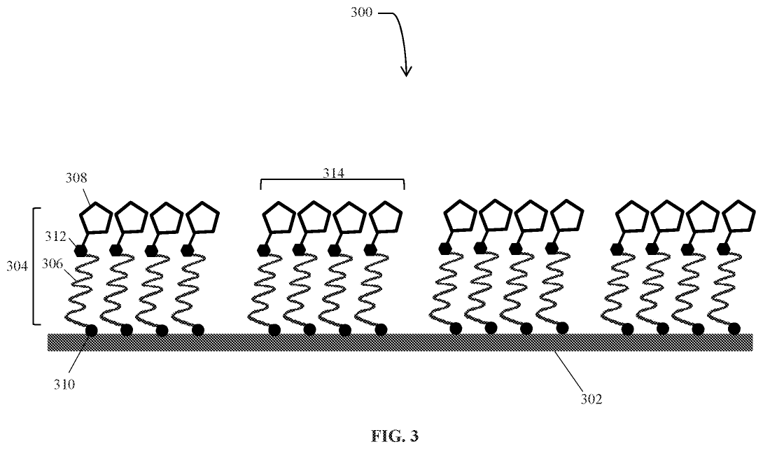

FIG. 3 shows a microarray 300 according to an example embodiment, the microarray 300 including a plurality of discrete regions 314, where each of the discrete regions 314 includes a coated substrate that includes a substrate 302 and a polymer layer 304 attached to substrate 302, where polymer layer 304 includes one or more polymers 306, one or more azlactone functional groups 308 attached to a first end of each of the one or more polymers 306 through a linker group 312, and one or more azide groups 310 attached to a second end of each of the one or more polymers 306, where the one or more azide groups 310 attach polymer layer 304 to substrate 302.

Example embodiments of the present invention also provide methods of preparing a bifunctional polymer, where a polymer, such as PEG, is functionalized with azlactone and azide groups.

According to example embodiments of the present invention, for example as described with respect to Examples 1 and 2 below, a method of preparing a bifunctional polymer includes: (a) providing a polymer, where the polymer contains one or more carboxylic acid groups or activated ester groups attached to a first end of the polymer and one or more azide groups attached to a second end of the polymer; (b) converting at least one carboxylic acid group or activated ester group into an aryl or vinyl halide group; and (c) attaching vinyldialkyl azlactone to the aryl or vinyl halide group. The polymer can be PEG. The vinyldialkyl azlactone can be attached to the aryl or vinyl halide group through a coupling reaction using a palladium catalyst, one or more solvents, and a base. The palladium catalyst can be selected from a group consisting of palladium (II) quinoline-8-carboxylate, palladium (II) chloride, palladium (II) bromide, palladium (II) acetate, palladium (II) acetoacetate, bis(dibenzylideneacetone)palladium(0), tris(dibenzylideneacetone)dipalladium(0), palladium (II) trifluoroacetate, allylpalladium (II) chloride dimer, bis(triphenylphosphine)palladium(II) dichloride, dichlorobis(tricyclohexylphosphine)palladium(II), and [1,1′-bis(di-tert-butylphosphino)ferrocene]dichloropalladium(II). The one or more solvents can be selected from a group consisting of dimethylformamide, dimethylsulfoxide, toluene, tetrahydrofuran, dioxane, dichloromethane, acetonitrile, and alcohol. The base can be selected from a group consisting of potassium carbonate, sodium carbonate, triethylamine, N,N-diisopropylethylamine, 4-(dimethylamino)pyridine, potassium tert-butoxide, and sodium tert-butoxide.

Example embodiments of the present invention also provide methods for functionalizing a surface with azlactone functionalized polymers, such as PEG. According to an example embodiment of the present invention, for example as described with respect to Example 3 below, there is provided a “top down” method of attaching to a substrate a bifunctional polymer that includes a polymer, one or more azlactone functional groups attached to a first end of the polymer, and one or more azide groups attached to a second end of the polymer, the method including: (a) providing a substrate containing alkyne functional groups; (b) providing a mixture that includes the bifunctional polymer, one or more solvents, a catalyst, a base, and a reducing agent; and (c) contacting the substrate with the mixture.

In the top down approach, the azlactone group and azido group are introduced to the ends of the polymer chain first, and then the polymer is attached to the surface containing alkyne groups via “click” reaction. This top down approach permits better control over the degree of functionalization, ensuring that each polymer chain has azlactone functionality. The substrate can be metal, metal oxide, silica, glass, carbon, or plastic. Methods of introducing alkyne functionality onto such surfaces are known. For example, Achatz et al., “Colloidal silica nanoparticles for use in click chemistry-based conjugations and fluorescent affinity assays,” Sensors and Actuators B: Chemistry, 150(1):211-19 (2010), discloses a method of decorating silica nanoparticles with O-(propargyl)-N-(triethoxysilylpropyl) carbamate. The polymer can be PEG or polysaccharides. The one or more solvents can be selected from a group consisting of dimethylformamide, dimethylsulfoxide, toluene, tetrahydrofuran, dioxane, acetonitrile, and water. The catalyst can be either a copper (II) salt or copper (I) salt (including copper sulfate, copper bromide, and copper iodide) or a ruthenium catalyst (including pentamethylcyclopentadienylbis(triphenylphosphine)ruthenium(II) chloride, pentamethylcyclopentadienyl(cyclooctadienyl)ruthenium(II) chloride, and pentamethylcyclopentadienyl(norbornadiene)ruthenium(II) chloride). The base can be selected from a group consisting of triethylamine, N,N-diisopropylethylamine, 4-(dimethylamino)pyridine, pyridine, quinolone, phenanthroline, and imidazole. The reducing agent can be selected from a group consisting of sodium ascorbate, tris(triazole)amine, and hydroquinones. Contacting the substrate with the mixture can be done at room temperature or elevated temperatures, such as from about 23 to 150° C., and the amount of reaction time permitted for contacting the substrate with the mixture correlates with the resulting density of PEG coating.

According to an alternative example embodiment of the present invention, a “bottom up” method of functionalizing a surface with azlactone includes providing a polymer, where the polymer contains one or more carboxylic acid groups attached to a first end of the polymer and one or more attachment groups attached to a second end of the polymer; attaching the polymer to the surface through the attachment group; and then converting the carboxylic acid group into azlactone. The polymer can be PEG. This bottom up approach permits the use of more alternative chemistries for attachment of the polymer to the surface, since the surface attachment is no longer required to be orthogonal to azlactone chemistry. In the bottom up approach, the attachment group of the polymer can be azide, phosphonic acid, carboxylic acid, silane, thiol, amine, hydrazine, alkyne, or catechol.

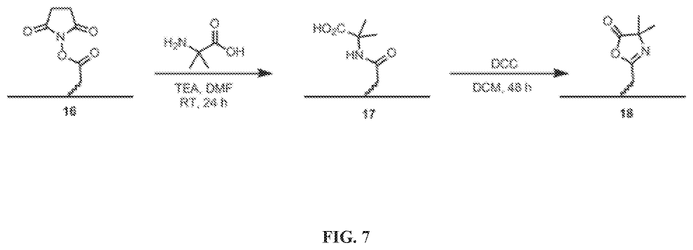

According to an example embodiment of the present invention, as shown in FIG. 7, an NHS-functionalized surface (16) is converted into an azlactone-functionalized surface (18) through solid phase synthesis. The NHS-functionalized surface is reacted with 2-methylalanine in the presence of a base (for example, trimethylamine (TEA)) and a solvent (for example, dimethylformamide (DMF)), resulting in intermediate structure (17). Intermediate structure (17) undergoes a cyclization reaction in the presence of a coupling reagent (for example, a carbodiimide, such as N,N′-dicyclohexylcarbodiimide (DCC)) and a solvent (for example, dichloromethane (DCM)), resulting in azlactone-functionalized surface (18).

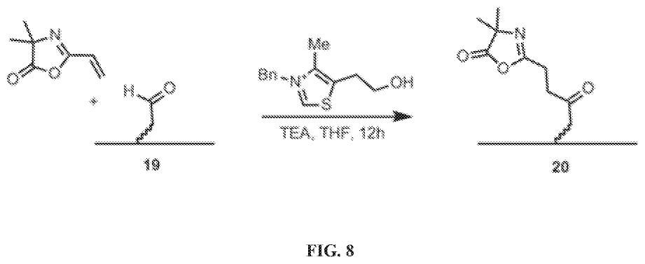

According to another example embodiment of the present invention, as shown in FIG. 8, an aldehyde-functionalized surface (19) is converted into an azlactone-functionalized surface (20) through solid phase synthesis. The aldehyde-functionalized surface (19) is reacted with vinyldialkyl azlactone in the presence of a catalyst (for example, 3-Benzyl-5-(2-hydroxyethyl)-4-methylthiazolium chloride), a base (for example, TEA), and a solvent (for example, tetrahydrofuran (THF)), resulting in the azlactone-functionalized surface (20).

A substrate with azlactone functional groups provides certain advantages, including a wider range of reaction conditions compatible with the substrate. Due to hydrolytic stability of azlactone groups, the substrate will tolerate a variety of buffers, pH levels, and other environmental conditions (including temperature and humidity), thus making it easier for a user to optimize biomolecule attachment chemistry and achieve improved signal intensity and uniformity.

For example, when using the spotting method to produce protein arrays on solid substrates, a drop of protein solution is dispensed onto the substrate and allowed to dry. The drying process influences the signal intensity and uniformity, where a slower drying process should improve conjugation efficiency (since there is more time for the reaction to happen) as well as minimize the coffee ring effect, where a circular outer perimeter contains higher protein concentration than the center. However, with groups such as NHS, activated esters, epoxides, and aldehydes, water hydrolysis competes with conjugation reaction, and therefore faster drying is preferred at the expense of better intensity and uniformity. The robustness of azlactone groups in aqueous solution allows for a slower drying process to achieve both optimal signal intensity and uniformity.

As another example, continuous-flow microprinting is another method of producing protein arrays. Continuous-flow microprinting provides longer contact of a functional substrate with a protein solution without drying and can be utilized to its full potential if a substrate has hydrolytically stable functional groups on the surface.

Example embodiments of the present invention also provide methods of using an azlactone functionalized substrate to conjugate biomolecules of interest.

According to an example embodiment of the present invention, a method of isolating biomolecules of interest includes: (a) providing a functionalized substrate that includes a substrate, one or more polymers, one or more azlactone groups attached to a first end of each of the one or more polymers, and one or more azide groups attached to a second end of each of the one or more polymers, where the one or more azide groups attach each of the one or more polymers to the substrate; (b) providing an aqueous solution that contains the biomolecules of interest; and (c) contacting the functionalized substrate with the aqueous solution for a period of time, where during the period of time, the biomolecules of interest attach to the azlactone groups. The aqueous solution can include an additive (such as glycerol, oligoethylene glycol, polyethylene glycol, surfactants, polyvinylalcohol, sugars, organic solvents, and inorganic salts), and can have a pH level in a range of from about 2 to about 10. Contacting the functionalized substrate with the aqueous solution can be achieved by jet printing, pin printing, quill printing, biological laser printing, capillary-based fluidics, or immersion. The period of time can be from several seconds to several hours. Additionally, biomolecules can be expressed and captured on the substrate in-situ using in vitro transcription and translation technology.

The above description is intended to be illustrative, and not restrictive. Those skilled in the art can appreciate from the foregoing description that the present invention may be implemented in a variety of forms, and that the various embodiments can be implemented alone or in combination. Therefore, while the embodiments of the present invention have been described in connection with particular examples thereof, the true scope of the embodiments and/or methods of the present invention should not be so limited since other modifications will become apparent to the skilled practitioner upon a study of the drawings, specification, and the following examples and claims.

The following are examples which illustrate specific methods without the intention to be limiting in any manner. The examples may be modified within the scope of the description as would be understood from the prevailing knowledge.

### EXAMPLES

### Example 1—Synthesis of Azido-PEG-Azlactone, Converting Carboxylic Acid Group into an Aryl Halide Group

A mixture of N3-PEG1k-CO2H (200 mg, 0.20 mmol, 1.0 equiv) (where 1k in N3-PEG1k-CO₂H represents a number of PEG units that provides an average molecular weight of approximately 1,000 g PEG per mole of the respective polymer and where “x” in Scheme (1) below represents a number of PEG units that can be any whole positive number, 1 or more, such as a number of PEG units that provides an average molecular weight of approximately 1,000 g PEG per mole of the respective polymer), 1-ethyl-3-[3-dimethylaminopropyl] carbodiimide hydrochloride (EDC) (37 mg, 0.24 mmol, 1.2 equiv), and iodoaniline (52 mg, 0.24 mmol, 1.2 equiv) in dry dichloromethane (DCM) was stirred under argon at room temperature for 12 hours. The reaction was quenched with water, and the organic layer was separated. The aqueous layer was extracted with DCM three times, and combined organic portions were dried over magnesium sulfate (MgSO₄). The solution was concentrated under reduced pressure to a crude oil, which was purified by silica gel column chromatography (0-10% methanol/DCM) to yield a soft white solid (225 mg, 82%).

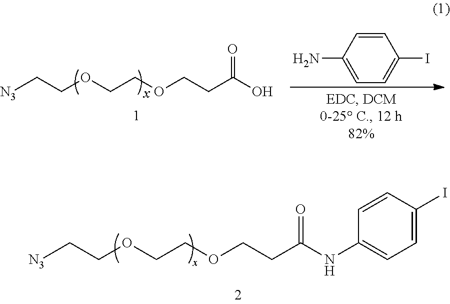

The structure of the resulting product was determined using proton nuclear magnetic resonance (NMR) spectroscopy, with the resulting NMR spectrum: ¹HNMR (600 MHz, CDCl3) δ=8.82 (bs, 1H), 7.58 (d, J=9.1 Hz, 2H), 7.39 (d, J=9.1 Hz, 2H), 3.81 (t, J=5.9 Hz, 2H), 3.70-3.59 (m, 96H), 3.38 (t, J=5.4 Hz, 2H), 2.63 (t, J=5.4 Hz, 2H) ppm; ¹³C NMR (125 MHz, CDCl3) δ=186.8, 137.8, 136.2, 123.2, 122.2, 82.9, 70.7, 38.1 ppm. The retention factor (R_{f}) of the product in thin layer chromatography (TLC) is as follows: TLC R_{f}=0.5 (10% MeOH/DCM).

### Example 2—Synthesis of Azido-PEG-Azlactone, Attaching Vinyldialkyl Azlactone to the Aryl Halide Group

Palladium catalyst “Quin₂Pd” was synthesized according to a procedure such as that described in Cui et al., “Pd(quinoline-8-carboxylate)₂ as a Low-Priced, Phosphine-Free Catalyst for Heck and Suzuki Reactions,” Journal of Organic Chemistry, 72:9342 (2007).

A 10 mL flame-dried Schlenk flask was charged with N3-PEG1k-iodoanilide (200 mg, 0.152 mmol, 1.0 equiv) (where 1k in N3-PEG1k-iodoanilide represents a number of PEG units that provides an average molecular weight of approximately 1,000 g PEG per mole of the respective polymer and where “x” in Scheme (2) below represents a number of PEG units that can be any whole positive number, 1 or more, such as a number of PEG units that provides an average molecular weight of approximately 1,000 g PEG per mole of the respective polymer) and Quin-Pd (3.2 mg, 0.0072 mmol, 0.05 equiv) and purged with argon. Dry dimethylformamide (DMF) (0.2 M, 0.8 mL), triethylamine (100 μL, 0.076 mmol, 5.0 equiv, dry and freshly distilled over CaH₂), and vinyl azlactone (58 μL, 0.456 mmol, 3.0 equiv) were added and the system was sealed. The reaction mixture was heated at 130° C. for 3 hours, turning a dark brown. It was then cooled to room temperature and concentrated under reduced pressure. The crude residue was dissolved in DCM and dry loaded onto Celite, which was applied to a silica column. Purification by column chromatography (0-20% methanol/DCM) yielded 156 mg.

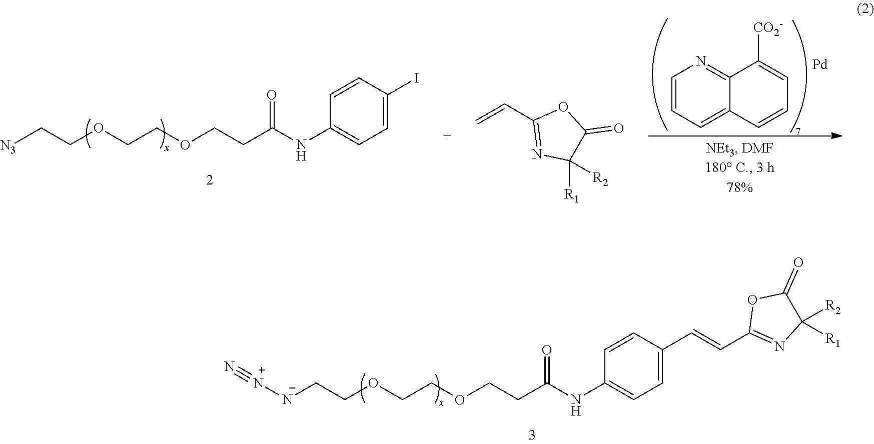

The structure of the resulting product was determined using proton NMR spectroscopy, with the resulting NMR spectrum: ¹H NMR (600 MHz) δ=9.10 (bs, 1H), 7.67 (d, J=8.0 Hz, 2H), 7.43 (d, J=8.0 Hz, 2H), 7.41 (d, J=15.4 Hz, 1H), 6.47 (d, J=16.5 Hz, 1H), 3.81 (t, J=6.0 Hz, 2H), 3.64-3.57 (m, ˜107H), 2.67 (t, J=5.2 Hz, 2H), 1.46 (s, 6H) ppm; ¹³C NMR (125 MHz) δ=181.0, 170.5, 159.6, 142.3, 140.7, 128.6, 120.1, 112.0, 70.5, 67.2, 46.4, 38.0, 24.9 ppm. The retention factor of the product in thin layer chromatography is as follows: TLC R_{f}=0.4 (10% MeOH/DCM).

### Example 3—Attaching Azido-PEG-Azlactone onto Alkyne-Functionalized Surface Via “Click” Reaction

To a solution (3 mg/mL) of azide-PEG-azlactone (where “x” in Schemes (3) and (4) below represents a number of PEG units that can be any whole positive number, 1 or more, such as a number of PEG units that provides an average molecular weight of approximately 1,000 g PEG per mole of the respective polymer) in 5 mL dimethyl sulfoxide (DMSO) and 5 mL deionized (DI) water was added 0.52 mL of 1.0 mM stock click chemistry solution (1.5 mg CuSO₄.5H₂O, 6 mg sodium ascorbate, 4 μL triethylamine, 3 mL DMSO, and 3 mL DI water). The slides functionalized with alkyne groups were immersed in this solution with gentle shaking for 12 hours at room temperature. The PEGylated slides were rinsed with DI water and spun dry.

Alternatively, PEG-azlactone was introduced onto a surface by pre-functionalizing the surface with azide groups, then coupling the surface with alkyne-PEG-azlactone via “click” reaction.

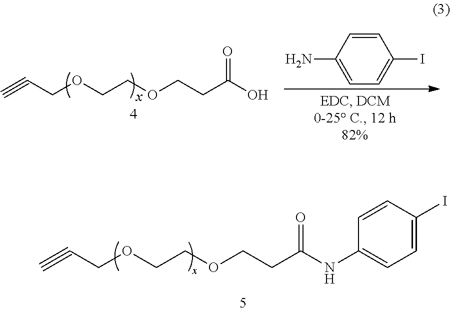

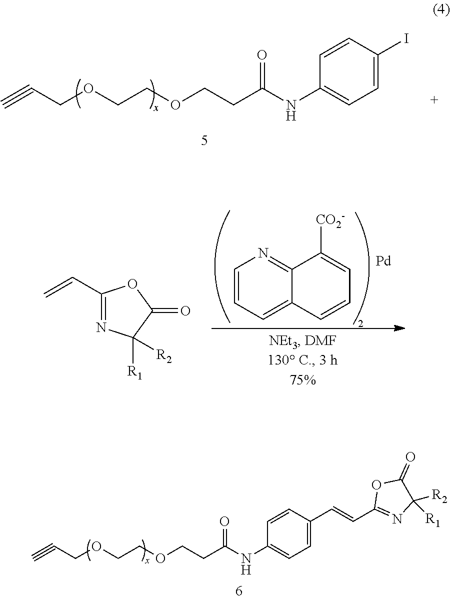

### Example 4—Synthesis of Polysaccharides Modified with Azlactone and Azide Functional Groups

As illustrated below, polysaccharides were oxidized to produce carboxylic acid groups suitable for further functionalization with azlactone. Some of the primary alcohols on polysaccharide chain (structure 7) were converted into carboxylic acid groups either using oxygen over platinum or using TEMPO ((2,2,6,6-Tetramethylpiperidin-1-yl)oxyl or (2,2,6,6-tetramethylpiperidin-1-yl)oxidanyl) as a catalyst and sodium hypochlorite (NaOCl) in basic pH, resulting in structure (8) (see Cumpstey, I., ISRN Organic Chemistry, Article ID 417672 (2013)). The carboxylic acid groups were then converted into aryl or vinyl halide (bromide or iodide) functionality, resulting in structure 9, followed by attachment of vinyldialkyl azlactone via palladium-catalyzed coupling to produce the desired azlactone-functionalized material (structure 10).

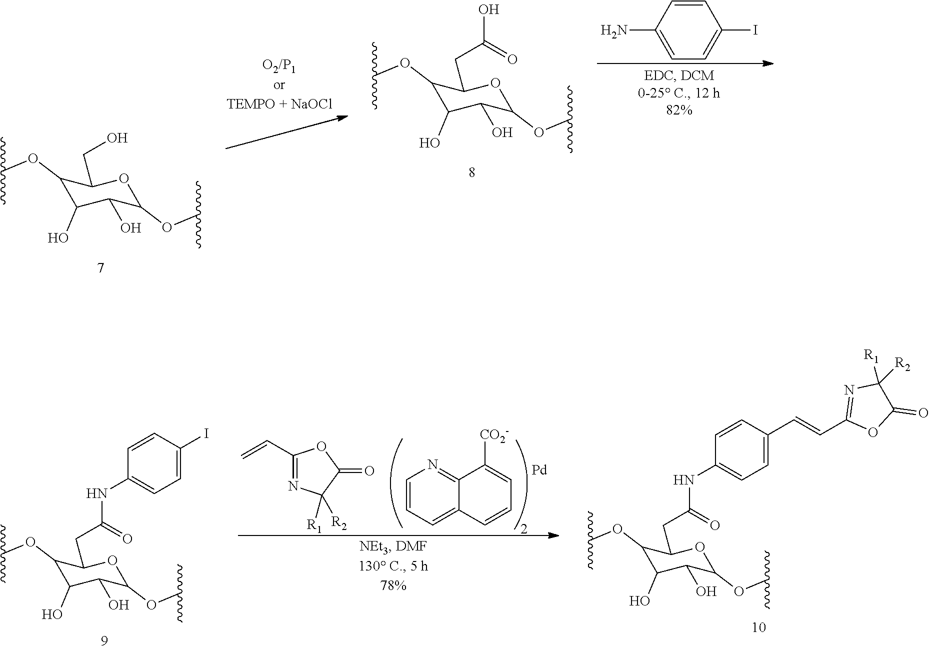

Alternatively, as illustrated below, periodate was used to achieve cleavage of 1,2 diols of the polysaccharide chain (structure 7) and introduce carboxylic acid functionality into the polymer chain, resulting in structure 11. Structure 11 was then reacted with iodoaniline, resulting in structure 12, which was then reacted with vinyldialkyl azlactone to produce the desired azlactone-functionalized material (structure 13).

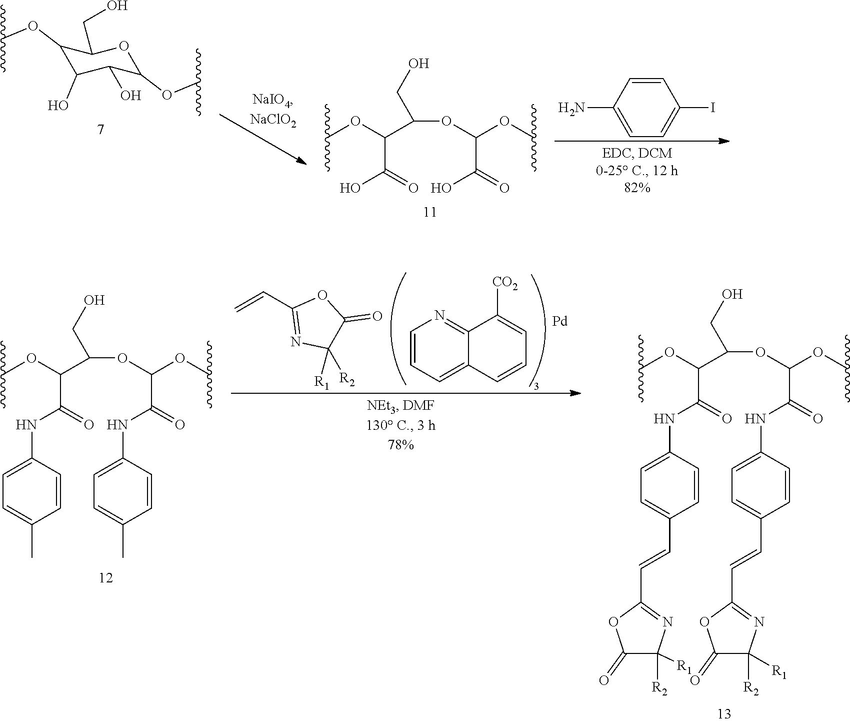

As illustrated below, azide functionality was introduced into the polysaccharide chain (structure 7) by converting some of the primary alcohols of the polysaccharide chain into bromides (for example, using N-bromosuccinimide (NBS) and triphenylphosphine (PPh)) or tosyl groups to give structure 14 (by reacting it with tosyl chloride (TsCl)) followed by reaction with sodium azide (NaN3) to produce structure 15 (see Cumpstey, I., ISRN Organic Chemistry, Article ID 417672 (2013)). This method can be combined with either of the two prior methods of synthesizing azlactone functionalized polysaccharides to yield a polysaccharide chain containing both azlactone and azide groups. Since only a few of the primary alcohols on a polysaccharide chain are modified into azlactones, the other alcohols are available for conversion into azides.

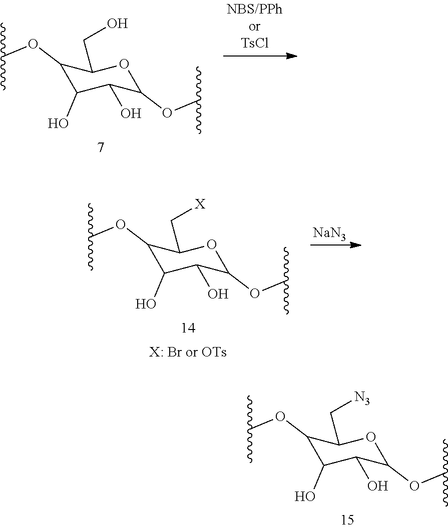

### Example 5—Attaching Protein to Substrate Coated with Azido-PEG-Azlactone Via Pin Printing

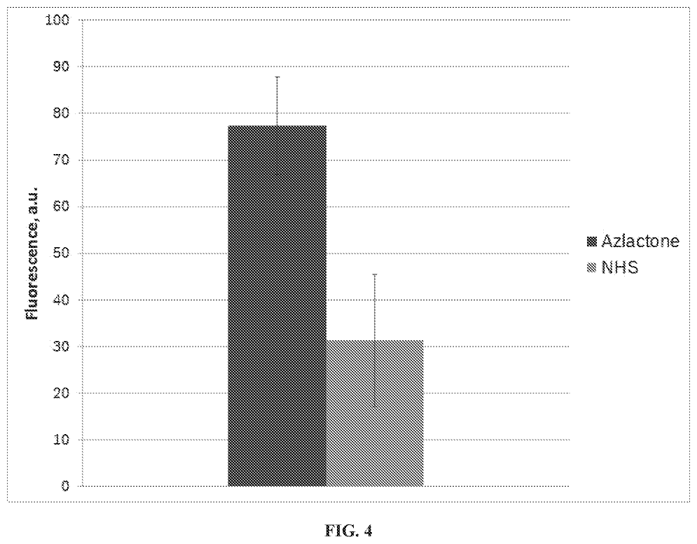

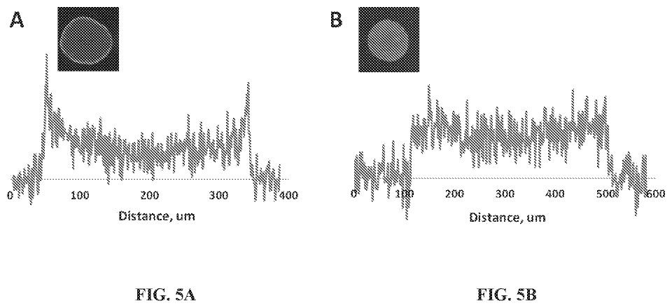

200 mg/mL solution of Green Fluorescent Protein (GFP) containing 0.1% of polyvinyl alcohol in phosphate buffered saline (PBS) was spotted onto the substrate using a pin printing method at room temperature and relative humidity of 55%. The spots were allowed to dry for 12 hours in a desiccator, and then the slides were rinsed with PBS-Tween and PBS to remove unbound protein. Relative amounts of GFP covalently attached to the substrate were determined by measuring the fluorescent intensity of the protein on the surface. The fluorescence intensity of azlactone-functionalized slides was 2.5 times higher compared to NHS ester-functionalized slides (see FIG. 4). Morphology of the slides was compared by looking at the fluorescence intensity across the spots. Protein spots on azlactone-functionalized surfaces were uniform (FIG. 5B), while spots on NHS-functionalized surfaces displayed characteristic coffee ring morphology (see FIG. 5A).

### Example 6—Protein Conjugation Via Immersion

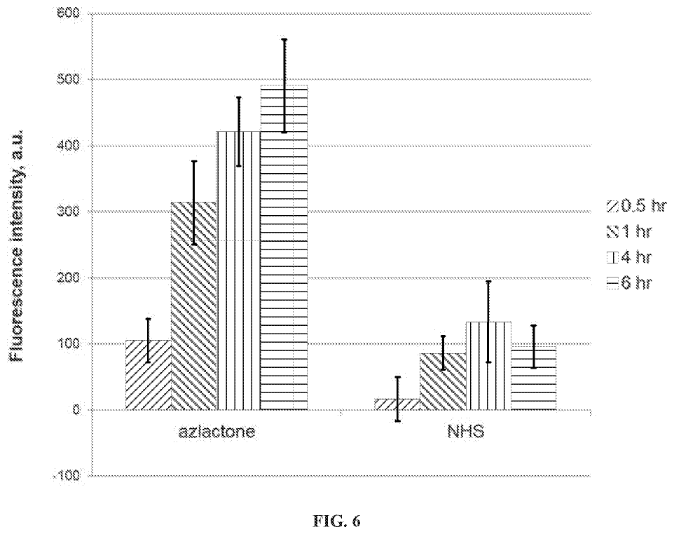

Glass substrates functionalized with azide-PEG-azlactone and azide-PEG-NHS containing the same density of functional groups on the surface were exposed to a solution of Immunoglobulin G (IgG) in phosphate buffered saline (PBS) at pH 7.4 for up to 6 hours. After removing the solution and rinsing the slides with PBS-Tween and PBS, the relative amounts of IgG covalently attached to the substrates were determined by measuring the fluorescent intensity of the protein on the surface. After 6 hours of incubation, fluorescence intensity of the azlactone-functionalized slides was 5 times higher compared to that of the NHS ester-functionalized slides (see FIG. 6).

1-23. (canceled)

24. A method of isolating a biomolecule of interest, the method comprising:
contacting a functionalized substrate with an aqueous solution comprising the biomolecule of interest for a period of time,
wherein the functionalized substrate is produced by a reaction between
a surface of the substrate, the surface being functionalized with an alkyne group, and
a polymer comprising an azide group and an azlactone group,
whereby the alkyne group of the surface and the azide group of the polymer react to attach the polymer to the substrate; and
wherein during the period of time, the biomolecule of interest reacts with the azlactone group of the polymer attached to the substrate, thereby covalently attaching the biomolecule of interest to the substrate.

25. The method of claim 24, wherein the aqueous solution further comprises an additive, wherein the additive is selected from a group consisting of glycerol, oligoethylene glycol, polyethylene glycol, surfactants, polyvinyl alcohol, sugars, organic solvents, and inorganic salts.

26. The method of claim 24, wherein a pH level of the aqueous solution is in a range of from about 2 to about 10.

27. The method of claim 24, wherein the contacting of the functionalized substrate with the aqueous solution is achieved by jet printing, pin printing, quill printing, biological laser printing, capillary-based fluidics, or immersion.

28. The method of claim 24, wherein the substrate comprises glass, silica, plastic, carbon, metal, or metal oxide.

29. The method of claim 24, wherein the polymer comprises a polyethylene glycol or a polysaccharide.

30. The method of claim 24, wherein the polymer comprises a polyethylene glycol having an average molecular weight of approximately 1,000 g/mole.

31. The method of claim 24, wherein the biomolecule is a protein.

32. The method of claim 24, wherein the substrate comprises a microarray having a plurality of discrete regions, each region being functionalized with the alkyne group.

33. The method of claim 24, wherein
the substrate comprises a glass surface functionalized with the alkyne group,
the polymer comprises a polyethylene glycol,
the biomolecule is a protein, and
the aqueous solution has a pH of about 7.4.

34. The method of claim 33, wherein the aqueous solution further comprises polyvinyl alcohol, and the contacting comprises pin printing the aqueous solution onto the functionalized substrate.

35. The method of claim 33, wherein the contacting comprises immersing the functionalized substrate in the aqueous solution for up to 6 hours.
